(19) United States
(12) Patent Application Publication
(10) Pub. No.: US 2018/0021080 A1
(43) Pub. Date: Jan. 25, 2018

(54) APPARATUS FOR TREATING SKIN BLEMISHES WITH COLD PLASMA

(71) Applicant: MDE WORKS S.R.L., Varedo (MB) (IT)

(72) Inventors: Eugenio FUMI, Varedo (MB) (IT); Danilo URBANI, Varedo (MB) (IT)

(21) Appl. No.: 15/550,058

(22) PCT Filed: Feb. 8, 2016
(86) PCT No.: PCT/IB2016/050634

§ 371 Date: Aug. 10, 2017

(30) Foreign Application Priority Data
Feb. 12, 2015 (IT) RM2015A000065

(51) Int. Cl. A61B 18/14; A61B 18/12

(52) U.S. Cl. CPC A61B 18/14; A61B 18/1233; A61B 2018/0047

(57) ABSTRACT

Disclosed is a medical apparatus including at least one electrode for generating cold plasma, the electrode including a tail and a head and being covered with isolating and biocompatible mineral material, the tail being the electrode portion to be connected with the corresponding handpiece and the head being the electrode portion to be faced towards the skin of the patient, the apparatus including at least one electrode including a head including a spherical end and/or at least one electrode including a head shaped to form a corkscrew-like element.

### FIELD OF THE ART

The present invention refers to the medical field. More in detail, the present invention refers to a new apparatus which uses cold plasma for applications in the dermatological field and in particular cosmetic medicine. Still more in detail, the present invention refers to a new apparatus which, by using cold plasma, allows correcting pathologies and/or blemishes of the skin tissues that are often due to aging.

### STATE OF THE ART

For years, methods have been known in cosmetic medicine for treating pathologies and/or blemishes of epidermal tissues. These include the following: laser therapy, based on the principle of light-stimulation of biological tissues, dermo-abrasion, which consists of a treatment adapted to improve the irregularities of the skin by eliminating the outermost layer of the skin in order to render it smoother and more even, and needling, which consists of a treatment of mechanical bio-remodeling of the face, for treating scars from acne and for stretch marks. All these technologies, while considered relatively effective for attaining the pre-established objects, nevertheless imply an invasive and sometimes painful approach. For example, the needling treatment consists of the micro-perforation of the skin with thin needles to a depth variable between two and three millimeters. In this manner, many thousands of micro-holes are made which trigger the natural skin healing process. The formation of natural collagen is induced along with the natural proliferation. The effect is that of a thickening of the skin, due to a tissue regeneration. Laser therapy instead shows the drawback of using relatively high temperatures, with consequent discomfort and reddening, for the obtainment of the vaporization of the skin surface layer that one intends to remove. For such purpose, the object of the present invention described in detail hereinbelow is that of correcting blemishes of the skin, generally due to pathologies of the skin or to aging phenomena, due to the use of a new and innovative apparatus that exploits cold plasma as curative agent. Said apparatus is in particular characterized in that it comprises components that not only facilitate the operator during treatment but also render said apparatus decidedly effective, being said components suitably designed and made for treating the skin with said cold plasma.

### DESCRIPTION OF THE INVENTION

The present description refers to a new and innovative apparatus which, by using cold plasma allows treating and correcting skin blemishes due, generally but not exclusively, to aging phenomena. Said apparatus is therefore used in the field of cosmetic medicine.

More in detail, the use of the present apparatus allows obtaining micro-holes in the stratum corneum of the epidermis, with characteristics of depth, diameter and density—being intended the number of micro-holes per unit of surface area—that are variable. All of this by operating at temperature values that are decidedly lower than those which characterize the conventional laser technology.

Advantageously the apparatus, by operating at relatively low temperatures, typically close to the body temperature, does not generate discomfort and reddening post-operation.

The apparatus for example comprises, in many embodiments thereof, at least one handpiece, comprising an electrode covered with a biocompatible mineral material, with a particular shape and with different curvature angles that ensure that the operator can treat both wide surfaces through the flat part, and deep wrinkles by means of the more curved part, by simply changing the tilt.

Between the handpiece and the skin surface, an RF voltage is created comprised between 2000-3000 V which ionizes the oxygen in the air—which at sea level represents about 21% of the air—by making O² react with O² to form O³+O⁻. The ozone that is formed, given that it is a powerful oxidizer, causes a vaporization of the affected tissue column.

The present apparatus allows actuating a method which, unlike needling and other mechanical methods which cross the skin barrier, has also advantageously proven to be a disinfection method, given that in fact the cold plasma is generally used for such purpose. The micro-holes therefore become sterile and one avoids the risk of bringing the bacteria normally present on the skin surface to a depth. Given that the action temperature is 45° C., there is no reddening nor discomfort sensation.

Advantageously, the use of the present apparatus also allows medications for topical and cosmetic use to cross the skin barrier. The stratum corneum of the skin is fact rich with lipids that nearly completely inhibit the penetration of drugs or cosmetics applied to the surface. The cold plasma used, comprising electrons with relatively high energy, is capable of causing the breakage of the chemical bonds of the lipids, disintegrating them, thus facilitating the active absorption of substances applied as cosmetics for several days, and of crossing the skin barrier without using more invasive methods such as injections, dermo-electroporation, ionophoresis etc.

Cold plasma is thus used in the present invention in a rejuvenation protocol. Typically, said protocol provides that, in a first step, micro-holes are generated with smaller diameter and greater depth so as to stimulate the fibroblasts and produce collagen and hyaluronic acid. The closure via repair of the micro-holes causes a tension of the skin, conferring tone to the same. In a second step, for electronic peeling, the frequency of discharges per second is instead increased (such discharges necessary for forming the plasma in the zone comprised between the handpiece and the treated skin surface). This step, which can also be repeated, serves for reducing blemishes, micro-scars from acne, micro-wrinkles, seborrheic dermatitis, and generally all those blemishes due to an increase of the stratum corneum and to chrono-aging and photo-aging.

In the aforesaid steps, the apparatus also allows following, if necessary, a capacitive diathermy: the present apparatus is in fact characterized not only by the possibility to produce (discharges with higher) power with RF with much higher frequencies than the RF apparatuses currently existing on the market, but also to measure, in real time and with high precision, the impedance of the treated tissue, allowing the transfer of the energy which is actually required and preventing the excessive overheating of the tissues. The apparatus, working at relatively high frequencies—comprised between 1 MHz and 15 MHz, preferably between 1.5-4 and 6.90 MHz, and typically 6.78 MHz—allows actuating a diathermy that stimulates Heat Shock Proteins, in particular HSC 70 and HSC 90, which come to restore and substitute the protein structures that were altered due to thermal damage or by free radicals. The administration of the capacitive diathermy occurs through a further ergonomic handpiece, with heads of various diameters, adapted to work both in wider zones, with a greater water content, hence gluteus, inner thighs, arms and abdomen, and in smaller, more delicate zones that require more precise movements, such as the face, neck etc. The effect resulting from the use of the apparatus is the gradual contraction, without superficial or deep alteration of the collagen fibers of the treated tissue, thus allowing greater support and compaction of the zones subjected to sagging.

The present apparatus is therefore a versatile instrument that also allows the integration of instrumentation for use in the action of the diathermy with low and high penetration as a function of the applied frequency.

The employed frequency preferably varies between 1.5 MHz and 6.780 MHz. The apparatus is provided with precision adjustments for all electrical parameters such as the peak power, the repetition frequency and the duty cycle of the pulses set by the operator. The apparatus comprises an RF power generator of (maximum) 200 W over 100 ohm continuously adjustable up to about 1 Watt. The frequencies are generated with a digital synthesizer, and can be selected in accordance with therapeutic protocols. The output power is measured with a Watt meter so as to always have the actual value of the applied power.

The apparatus, described in detail hereinbelow, further comprises accessories such as: a mass plate, at least one electrode carrier handpiece for resistive and capacitive diathermy with relative electrodes of various diameters, and is characterized in that it comprises at least one handpiece for plasma with isolated electrodes of various geometric forms, covered with biocompatible isolating mineral material such as pure alumina deposited on metal.

### BRIEF DESCRIPTION OF THE DRAWINGS

FIG. 1 shows a block diagram of several of the components of the medical apparatus, object of the present industrial invention patent application. The block diagram shows that said apparatus comprises: a first unit 1 containing at least one color LCD display placed on the front panel with touch screen for machine operation; a second unit 2 comprising a microprocessor 11 with memory 10 for resident programs, the DDS 12, the modulator-attenuator 13, the amplifier 14 adapted to restore the losses of the modulator and a low-pass filter 15; a third unit 3 comprising a pre-driver and an RF broadband power amplifier for the signals coming from the second unit 2 up to a maximum power of over 200 watt, preferably 100 watt; a fourth unit 4 comprising RF power filters; a fifth unit 5 containing a measuring card in which the voltage V is measured along with the current I and relative phase, therebetween, of the radiofrequency signal; a sixth unit 6 placed at the output of the machine adapted for the galvanic isolation of the patient from the mass. Said unit 6 in fact comprises a high isolation 1:1 RF transformer 21, a set of HT photo couplers 22 and a high isolation DC-DC 23 for power supplying the electronics necessary for the services in the handpieces 18 having connection 19 to the apparatus, and the possible return plate 20 connected with the patient; an eighth unit 8 including a power supply unit which, by drawing the necessary power from the electricity network, provides for power supplying all the circuits of the machine; and a ninth unit 9 comprising the network switch, fuses, anti-disturbance filters. The apparatus also has a PC card (driver) 24 for managing the images and USB ports 25.

FIG. 2 shows a front view (FIG. 2 (a)), longitudinal section view (FIG. 2 (b)) and perspective view (FIG. 2 (c)) of an electrode 100 characterizing the medical apparatus, object of the present invention. More in detail, the figure shows that said electrode 100 substantially comprises two portions: the head 110 and the tail 120 of the electrode. The tail 120 represents the electrode portion to be inserted within the corresponding handpiece 18, while the head 110 represents the electrode portion that is actually “active”, i.e. the electrode portion 100 that when moved acts on the skin to be treated. If the treatment to be executed is to be performed on small portions of skin tissue, and for example provides for a considerably peeling or a treatment on cutaneous blemishes, the head 110 of the employed electrode has an end 130 with spherical profile, as in the figure in question.

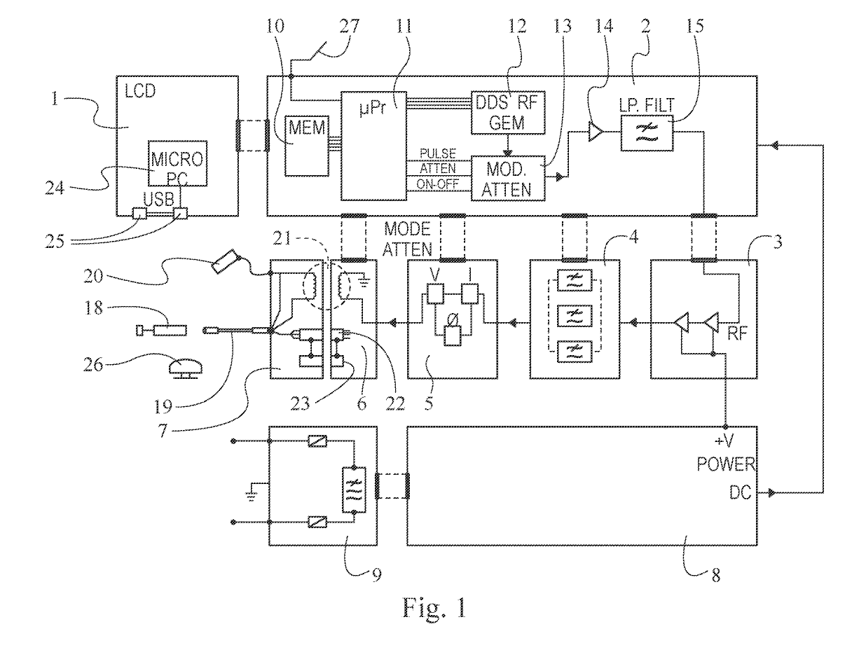

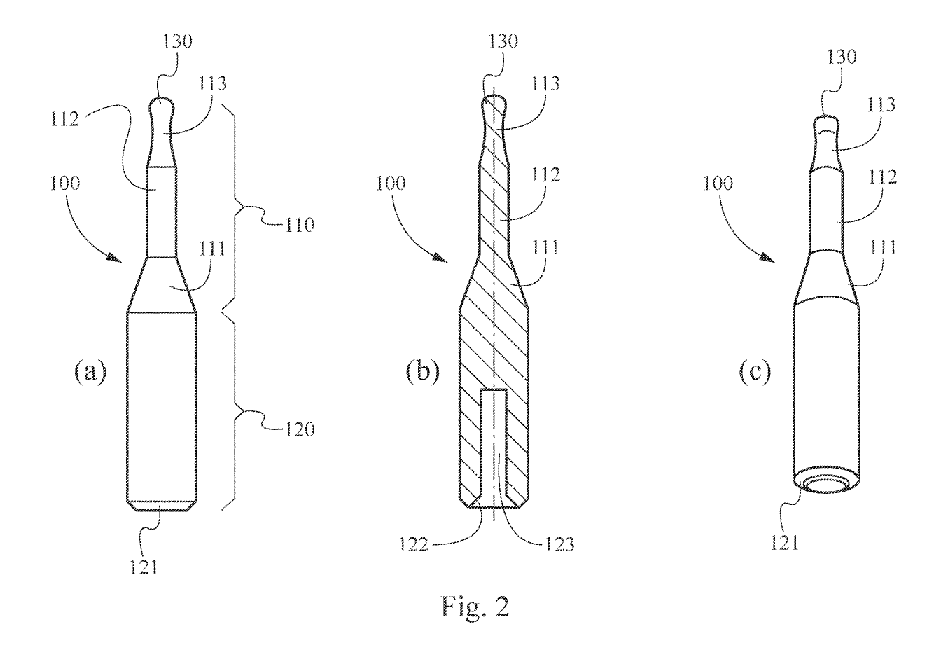

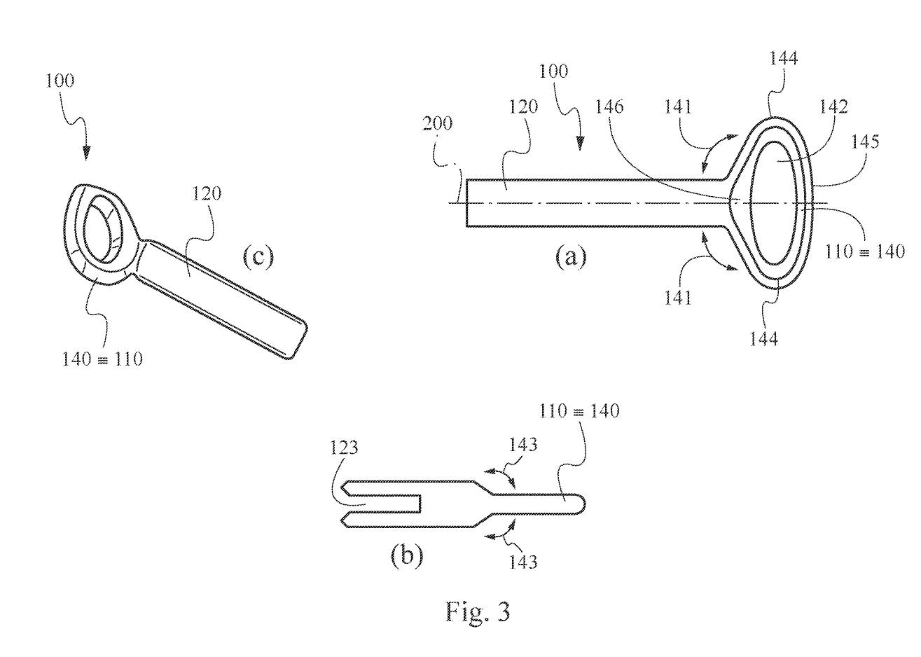

More in detail, FIG. 2 shows that the represented electrode 100 comprises, starting from the tail 120 and being directed towards the spherical end 130: a conical portion 111, a cylindrical central portion 112 having diameter of the transverse section analogous to that of the lower base of the cone represented by said conical portion 111, a catenoid-like pinched cylindrical portion 113, and said spherical end 130. The tail 120 instead has a hollow bottom 121 adapted to allow the connection of the electrode with the corresponding handpiece 18. Said hollow bottom 121 centrally has the flaring 122, from which cavity 123 departs, it too preferably but not necessarily cylindrical, whose length is extended for about half the length of said tail 120.

FIG. 3 shows a front view (FIG. 3 (a)), lateral view (FIG. 3 (b)) and perspective view (FIG. 3 (c)) of an electrode 100 comprising a head 110 shaped to form a corkscrew-like element 140. The electrode 100 represented in the figure in question can be used, in accordance with the treatment to be executed, in two spatial configurations: one in which the hole 142, defined by the head 110 shaped to form a corkscrew-like element 140, is oriented nearly parallel to the skin surface to be treated (FIG. 3 (a)), the other in which said hole 142 is oriented nearly orthogonal to the skin surface to be treated. The latter spatial configuration for example corresponds with the condition in which it is necessary to treat deep skin zones, while the former corresponds with the condition in which it is necessary to treat wide surfaces.

More in detail, said head 110 shaped to form a corkscrew-like element 140 is defined by a closed curve that is symmetric with respect to the longitudinal axis 200 of the electrode and forms, when arranged parallel to the surface to be treated, two angles 141 with the tail 120 of the same electrode. When, instead, the head 110 shaped to form a corkscrew-like element 140 is arranged orthogonal to the skin surface to be treated, said head shows two other symmetric angles 143 that are formed with said tail 120. Said head 110, when shaped to form a corkscrew-like element 140, also has: at least two lateral curvatures 144, at least one upper curvature 145, and at least one lower curvature 146.

### DESCRIPTION OF THE PREFERRED EMBODIMENTS

The present industrial invention patent application refers to a new apparatus to be used in the dermatology field, and specifically in the treatment of zones of the body affected by pathologies and/or blemishes such as, by way of a non-limiting example, aging of the face, extended striae (stretch marks), tissue relaxation, irregularity of surfaces due to scars from acne or from trauma, etc. The improvement of the treated epidermal tissue is progressive and effective, especially when the treatment using the present apparatus is repeated at intervals of about a week for about six-ten sessions. The present apparatus allows attaining the aforesaid results, pertaining to the treatment of pathologies or the correction of blemishes, by using particular electrodes for cold plasma generated by means of high pulsed voltage or radiofrequency with high pulsed voltage. More in detail, the apparatus allows the production of a succession of electrical discharges of very brief duration through a dielectric, placed between the electrode of the apparatus and the skin of the patient, so that a discharge of “corona” type is generated. The dielectric prevents the discharge from being transformed into an arc by keeping it cold. In all embodiments thereof, the present apparatus is substantially characterized in that it comprises an electrode/electrodes suitably studied and designed for the type of body surface, pathology and/or blemish to be treated by generating cold plasma. All this by making the treatment associated with such apparatus decidedly specific and effective. The technology associated with such apparatus allows the selective removal of the lipid stratum corneum of the skin, due to the strong action of the high-energy electrons which break the chemical bonds of the lipids themselves, disintegrating them. In addition, a strong germicide action can be found, due to the oxidizing effect of the ozone and due to the UV that are generated during the myriad electrical micro-discharges that take place between the electrode and the surface of the skin. All of the above determining an ablation action that is painless and without trauma.

The obtained discharges also produce a series of micro-holes which affect a thickness of several hundred microns of the dermis, whose subsequent natural repair produces a compaction of the treated tissue. The distance between these holes can be varied by means of the pulse repetition frequency, while their size and depth vary as a function of the supplied energy or peak power and pulse duration. Visually, micro-sparks with blue-violet color can be observed during treatment between the electric layer of the handpiece and the surface of the skin, such micro-sparks tending towards orange color when power is increased in order to have deeper micro-holes. These micro-holes do not affect the entire surface of the treated skin but a relatively small percentage, even if in large quantity. In such a manner, the alternation of perforated skin zones and intact tissue zones is determined, these function as a biological reserve for a quick healing without recovery times. In addition, the slight heat increase encountered by the skin areas interposed between the single micro-holes activates the cellular metabolism, inducing the repair and tissue remodeling processes; this effect is due to the activation of the fibroblasts with the production of hyaluronic acid, elastin and components of the intercellular matrix and type 3 collagen.

The medical apparatus, object of the present description, is also characterized in that, in some embodiments, it comprises components that allow supplementing the plasma treatment with the use of diathermy with low and high penetration as a function of the frequency. As already mentioned, the employed frequency varies between 1 MHz and 15 MHz, preferably between 1.5 MHz-4-6.8 MHz. More in detail, in some embodiments the apparatus comprises specific handpieces for diathermy, in which it is possible to measure, in real time and without contact, the temperature of the dermis, executed by means of infrared through a small window at the center of the application electrode/handpiece. This reading allows an accurate control of the dermal temperature reached and hence allows not exceeding 45°, deemed damaging for the therapy itself. It is also possible to limit the temperature of the dermis to a preset value, so as to automate the supply of the necessary power.

The present medical apparatus first of all externally comprises a mechanical structure for containment and electromagnetic screening, adapted to not irradiate the undesired disturbance signals. At the apparatus interior, there is a series of functional units, each comprising specific components. More in detail the apparatus internally comprises: a first unit 1 containing a color LCD display placed on the front panel with touch screen for the operation of the machine itself; a second unit 2 represented by a logic card containing: a microprocessor 11 which controls the functions of the machine with its memory 10 for resident programs, the DDS 12 which provides for generating the necessary RF signals as a function of the set therapies, the modulator-attenuator 13 which, controlled by the microprocessor 11, generates the pulses, the ON-OFF function and the adjustment of intensity of the output RF of the entire machine. An amplifier 14 restores the losses of the modulator, a low-pass filter 15 limits the band to that of interest. The apparatus then internally comprises a third unit 3 comprising a pre-driver and a RF broadband power amplifier for the signals coming from the second unit 2 up to a power of 200 watt; a fourth unit 4 comprising at least one filter, preferably three RF power filters, inserted by the microprocessor depending on the frequency generated by the DDS and used for the different therapies; a fifth unit 5 containing a measuring card where the voltage V is measured along with the current I and relative phase, therebetween, of the radiofrequency signal, and with these measurements the microprocessor is capable of calculating both the actual RF power supplied to the load and the actual and imaginary impedance of the load; a sixth unit 6 placed at the output of the machine, adapted to provide for both the galvanic isolation of the load (of the patient) from the mass, and the second isolation from the system of the machine itself. At its interior, there is a high isolation 1:1 RF transformer 21, a set of HT photo couplers 22, and a high isolation DC-DC 23 for power supplying the electronics necessary for the services in the handpieces 18; a seventh unit 7 represents the isolated side, hence the side of the patient, where all the handpieces 18 are connected with connection 19 to the apparatus for the treatments to be executed, and also the possible return plate 20 connected with the patient; an eighth unit 8 including a power supply unit which, by drawing the necessary power from the electricity network, provides for power supplying all the circuits of the machine; a ninth unit 9 comprising the network switch, fuses and anti-disturbance filters, medical standard, which provides for ensuring that the machine does not disturb and is not disturbed by spurious electrical signals.

The first unit 1, in addition to the LCD display, further comprises a PC card (driver) 24 for managing the images, the USB ports 25, and the possible Internet connection for upgrades and diagnostics. The accessory equipment further comprises electrode carrier handpieces for diathermy, with heads 26 of various diameters and at least one for plasma.

In the rear portion of the present apparatus, the following are also comprised: (the network input on the rear) network and switch input, box mass, protected service RS 232/485 connection, USB, ventilation output, pneumatic pedal control.

The second unit 2 further comprises the pedal input 27 adapted to activate plasma generation for the entire time that it is driven.

As mentioned several times in the course of the present description, the present invention refers to a medical apparatus which is characterized in that it comprises electrodes suitably studied, designed and achieved for treating and healing blemishes of the skin by means of cold plasma generation. The present application therefore also intends to claim the electrodes in question for use in the treatment of the aforesaid pathologies and/or skin blemishes. Said electrodes are more specifically made not only for cold plasma generation in proximity to the skin surface to be treated, but also for optimizing treatment effectiveness by assisting the operator in performing maneuvers during the same treatment. All this while ensuring that the patient is isolated from the discharges produced, thus avoiding the risk of burns. Said electrodes are in fact covered with a biocompatible isolating mineral material that is uniformly extended on the surface of the head of the electrode. Typically, but not limited thereto, pure alumina is deposited on metal. The goal is to obtain an electrode comprising a head covered with a uniformly distributed material that does not have pores, which cause isolation losses with consequent discharges that are damaging for the skin of the patient at high voltages. Said covering of the electrode is of great importance since it must ensure an isolation seal even in case of incorrect maneuvers by the operator, which would otherwise result in unaesthetic burns on the skin.

More in detail, the present apparatus is characterized in that it comprises at least one handpiece 18 comprising a corresponding electrode 100 for plasma. Said electrode 100, as already mentioned, is specific for the cold plasma treatment to be executed on the skin of the patient. For such purpose, it is profiled and shaped in order to make the treatment type effective and assist the operator in executing said treatment. The electrode 100 therefore has a geometric profile that is specific for the treatment to be executed. The present apparatus is therefore a decidedly versatile instrument since, in many embodiments thereof, it comprises a plurality of variously-profiled electrodes in accordance with the type of substrate and blemishes/pathology to be treated. More in detail the apparatus comprises an electrode in which two portions can be identified which, for the sake of simplicity, in the course of the present description will be indicated as head 110 and tail 120 of the electrode. The tail 120 represents the electrode portion to be inserted in the corresponding handpiece 18, while the head 110 represents the electrode portion that is actually “active”, i.e. the electrode portion that, when moved, acts on the skin to be treated. Still more in detail, if the treatment to be executed is to be performed on small portions of skin tissue and for example provides for a considerable peeling or a treatment on skin blemishes, the head 110 of the employed electrode will have an end 130 with spherical profile. If instead the treatment provides for the correction of skin blemishes involving relatively large skin portions, the apparatus will comprise an electrode 100 comprising a head 110 shaped to form a corkscrew-like element 140. These and other profiles can have the electrodes 100 comprised in the present apparatus; nevertheless, in the course of the present description, reference will be made to an electrode 100 comprising a head 110 with spherical end 130, and to an electrode 100 comprising a head 110 shaped to form a corkscrew-like element 140. Independent of the geometric profile of the ends of the heads 110, the tail 120 of the electrodes 100 can be the same for all the electrodes 100 comprised in the apparatus.

More in detail, the electrode 100 comprised in the present apparatus comprises, by way of a non-limiting example, a tail 120 with cylindrical profile. Said tail 120 has a hollow bottom 121 adapted to allow the connection of the electrode with the corresponding handpiece 18. Said hollow bottom 121 centrally has the flaring 122 having diameter comprised between 3 and 6 mm, preferably 4.5 mm at 90°, and from said flaring the cavity 123 departs, it too preferably but not necessarily cylindrical, whose length 1 is extended for about half the length of said tail 120. More in detail, said cylindrical cavity 123 has a transverse section with width comprised between 1.5 mm and 3.0 mm, preferably 2.3 mm. The length of said cavity 123 varies between 10 mm and 20 mm and is preferably 15 mm. The transverse section of the tail 120 has width comprised between 6 mm and 12 mm and is preferably 8 mm.

More in detail, by referring to an electrode 100 comprising a head 110 having spherical end 130, said head 110 comprises—starting from the tail 120 and being directed towards the spherical end 130—a conical portion 111, a cylindrical central portion 112 having diameter of the transverse section analogous to that of the lower base of the cone represented by said conical portion 111, a catenoid-like pinched cylindrical portion 113 having radius of curvature—being intended that of the catenary from whose rotation said pinched cylindrical portion 113 is obtainable—comprised between 12 mm and 14 mm, preferably 13.25 mm, and the spherical end 130 having a radius comprised between 1 and 2 mm, preferably 1.5 mm. The length of the entire electrode 100 is variable and is preferably comprised between 40 and 60 mm and is still more preferably 52 mm. The length of the tail 120 is preferably, but not necessarily, comprised between 20 mm and 30 mm and is preferably 25 mm, that of the conical portion 111 is preferably comprised between 2 and 12 mm and is still more preferably 7 mm, that of the cylindrical central portion 112 is preferably comprised between 6 mm and 16 mm and is still more preferably 11.31 mm, and the length of the catenoid-like pinched cylindrical portion 113 is preferably comprised between 3 mm and 13 mm and is still more preferably 8.69 mm.

As already mentioned, said electrode is particularly effective in the cold plasma treatment on relatively small skin portions. Otherwise, if the treatment provides that the electrode acts on larger surfaces, then it will be preferable for the present apparatus to comprise at least one electrode 100 comprising a head 110 shaped to form a corkscrew-like element 140. In this embodiment, the electrode 100 will preferably have a tail 120 with size analogous or similar to that described above for the electrode with spherical end 130. Different from the latter, said electrode with head 110 shaped to form a corkscrew-like element 140 comprises a head more specifically including various curved portions.

For the purpose of comprehension of the present description, it is suitable to specify that many different spatial orientations are possible for said electrode during its use, which are specific for the treatment to be executed. For the sake of simplicity, two spatial configurations of the electrode with corkscrew-like head 140 will be considered herein. A first spatial configuration, in which the head 110 shaped to form a corkscrew-like element 140 is to be directed such that the hole 142 defined by said corkscrew is nearly parallel to the skin—corresponding to the treatment condition of wide surfaces—and a second spatial configuration, in which said hole 142 is oriented nearly orthogonal with respect to the skin surface to be treated. The latter spatial configuration for example corresponds to the condition in which it is necessary to treat deep skin zones. More in detail, said head 110 shaped to form a corkscrew-like element 140 is defined by a closed curve that is symmetric with respect to the longitudinal axis 200 of the electrode 100 to which it belongs, and forms, when it assumes the aforesaid first spatial configuration, at the connection with the tail 120 of said electrode, two lateral symmetric angles 141 comprised between 100° and 115°, preferably 112.75°. When instead it assumes the aforesaid second spatial configuration, the head 110 shaped to form a corkscrew-like element shows two other front/rear symmetric angles 143 which are formed with said tail 120. More in detail, said angles are comprised between 130° and 140° and are preferably 135°. By arranging the electrode 100 in the aforesaid first spatial configuration and by referring to the curvatures presented by said corkscrew-like end 140, defining the aforesaid hole 142, the following can be identified: at least two lateral curvatures 144, being intended at least one for each side, at least one upper curvature 145, and at least one lower curvature 146.

A particular embodiment of the invention provides that the present medical apparatus comprises electrodes 100 with interchangeable heads 110 in accordance with the type of treatment to be executed.

As indicated several times in the course of the present description, it is very important that in all embodiments thereof and independent of the type of cold plasma treatment requested, the electrodes 100 are covered with a material that ensures the electrical isolation of the patient from the emitted discharges. All this in order to avoid the risk of accidental unaesthetic burns. For such purpose, the electrodes 100 in question are covered with specific isolated materials. In many embodiments thereof, said apparatus comprises electrodes 100 covered with alumina, while in other embodiments said electrodes are covered with silicon nitride.

Other embodiments of the invention provide, as already mentioned, that the present apparatus also comprises electrodes—with heads of various diameters—for diathermy.

1. Apparatus externally comprising: at least one mechanical structure for the containment and electromagnetic screening, and internally comprising: a first unit (1) comprising: at least one color LCD display with touch screen and accessible from a front panel of said apparatus; a second unit (2) comprising: at least one logic card in turn containing a microprocessor (11) with memory (10) for resident programs, at least one DDS (12) adapted to generate RF signals preset according to need, at least one modulator-attenuator (13) controlled by the microprocessor (11) and generating pulses, at least one ON-OFF function, at least one system for the adjustment of intensity of the output radiofrequency of the entire machine, at least one amplifier (14) adapted to restore the losses of said modulator-attenuator (13), and at least one low-pass filter (15) adapted to limit the band to the desired one, said radio frequencies being comprised between 1 MHz and 15 MHz; said medical apparatus further internally comprising a third unit (3) comprising: at least one pre-driver and at least one RF broadband power amplifier for the signals coming from said second unit (2) up to a power of 200 watts; a fourth unit (4) comprising: at least one RF power filter inserted by the microprocessor (11) depending on the frequency generated by the DDS (12); a fifth unit (5) comprising: a measuring card for measuring the voltage V and current I and relative phase, there between, of the RF signal, said measurements allowing the microprocessor (11) to calculate the actual radiofrequency power supplied to the load and the actual as well as the imaginary impedance of the load; a sixth unit (6) arranged at the output of said apparatus adapted to provide for the galvanic isolation of the load from the mass, said load being a patient, said unit (6) comprising: at least one high isolation 1:1 RF transformer (21), a set of HT photo couplers 22, and at least one high isolation DC-DC (23) for power supplying electrode carrier handpieces (18); a seventh unit (7) adapted for the connection, through connections (19), of corresponding electrode carrier handpieces (18), at least one return plate (20) connected to the load; an eighth unit (8) including at least one power supply unit for supplying all electrical circuits of said apparatus; and a ninth unit (9) comprising the network switch, fuses and medical standard anti-disturbance filters, said medical apparatus comprising at least one electrode (100) for generating cold plasma, said electrode (100) comprising a tail (120) and a head (110) and being covered with isolating and biocompatible mineral material, said tail (120) being the electrode portion to be connected with the corresponding handpiece (18) and said head (110) being the electrode portion (100) to be faced towards the skin of the patient, said apparatus comprising at least one electrode (100) comprising a head (110) including a spherical end (130) and/or at least one electrode (100) comprising a head (110) shaped to form a corkscrew-like element (140).

2. Apparatus according to claim 1, wherein the electrode (100) comprising the head (110) including the spherical end (130) comprises a cylindrically-shaped tail (120) having a hollow bottom (121), said bottom (121) centrally having a flaring (122) having diameter comprised between 3 mm and 6 mm at 90°, from said flaring (122) there departing an inner cavity (123) having length comparable to that of the half of the length of said tail (120), said length of the cavity (123) being comprised between 10 mm and 20 mm, said cavity (123) having a transverse section having width comprised between 1.5 mm and 3.0 mm, said tail (120) having width comprised between 6 mm and 12 mm, said electrode (100), starting from said tail (120) and moving towards said spherical end (130), further comprising: a conical portion (111), a cylindrical central portion (112) having diameter of the transverse section analogous to that of the lower base of the cone represented by said conical portion (111), a catenoid-like pinched cylindrical portion (113) having a radius of curvature comprised between 12 mm and 14 mm, and said spherical end (130), the latter having a radius of curvature comprised between 1 mm and 2 mm, said electrode (100) with spherical end (130) having an overall length comprised between 40 mm and 60 mm, said conical portion (111) having length comprised between 2 mm and 12 mm, said central cylindrical portion (112) having length comprised between 6 mm and 16 mm, said catenoid-like pinched cylindrical portion (113) having length comprised between 3 mm and 13 mm.

3. Apparatus according to claim 2, wherein the electrode (100) comprising the head (110) including the spherical end (130) comprises: the flaring (122) having a diameter of 4.5 mm at 90°, the inner cavity (123) having a width of 2.3 mm and length of 15 mm, the tail (120) having width of 8 mm and length of 25 mm, the conical portion (111) having a length of 7 mm, the central cylindrical portion (112) having a length of 11.31 mm, the catenoid-like pinched cylindrical portion (113) having a length of 8.69 mm and radius of curvature of 13.25 mm, the spherical end (130) having a radius of 1.5 mm, said electrode (100) with spherical end (130) having an overall length of 52 mm.

4. Apparatus according to claim 3, further comprising at least one electrode (100) comprising a tail (120) analogous to that of the electrode (100) comprising the spherical end (130), and a head (110) shaped to form a corkscrew-like element (140), the latter centrally defining the hole (142), said head (110) shaped to form a corkscrew-like element (140) defining, with the tail (120) of the same electrode in which it is comprised, the lateral symmetric angles (141) comprised between 100° and 115°, said angles being observable when said hole (142) is parallel to the skin of the patient, said head (110) shaped to form a corkscrew-like element (140) further defining with said tail (120) the front/rear symmetric angles (143) comprised between 130° and 140°, said angles being visible when said hole is orthogonal to the skin of the patient, said head (110) shaped to form a corkscrew-like element (140) further having, for each side, at least one lateral curvature (144), at least one upper curvature (145), and at least one lower curvature (146).

5. Apparatus according to claim 4, wherein the symmetric lateral angles (141) are 112.75° and in that the front/rear symmetric angles (143) are 135°.

6. Apparatus according to claim 1, further comprising on the rear portion of said apparatus: the network and switch input, box mass, protected service connection, USB, ventilation output, pneumatic pedal control (27).

7. Apparatus according to claim 1, wherein the generated radiofrequencies are comprised between 1.5 MHz and 6.87 MHz.

8. Apparatus according to claim 1, wherein the radiofrequencies have a frequency of 4 MHz.

9. Apparatus according to claim 1, further comprising electrodes (100) with interchangeable heads (110).

10. Apparatus according to claim 1, wherein the electrode/electrodes (100) is/are covered with alumina.

11. Apparatus according to claim 1, wherein the electrode/electrodes (100) is/are covered with silicon nitride.

12. Apparatus according to claim 1, further comprising in the first unit (1) at least one PC card driver (24) for managing the images, of the USB ports (25), of the possible Internet connection for upgrades and diagnostics; and further comprising, in the second unit (2), the pedal input (27) adapted to activate plasma generation.

13. Apparatus according to claim 1, further comprising electrode carrier handpieces for diathermy with heads (26) of various diameters.

14. Apparatus according to claim 1 for medical use.

15. Apparatus according to claim 1 for use in the cold plasma treatment of skin blemishes due to skin diseases and/or aging.

16. Electrode (100) comprising a tail (120) and a head (110) wherein the head (110) includes a spherical end (130) and comprising: a tail (120) with cylindrical shape having a hollow bottom (121), said bottom (121) centrally having the flaring (122) having diameter comprised between 3 mm and 6 mm at 90°, from said flaring (122) there departing the internal cavity (123) having a length comparable to that of half the length of said tail (120), said length of the cavity (123) being comprised between 10 mm and 20 mm, said cavity (123) having a transverse section having width comprised between 1.5 mm and 3.0 mm, said tail (120) having width comprised between 6 mm and 12 mm, said electrode (100), starting from said tail (120) and moving towards said spherical end (130), further comprising: a conical portion (111), a cylindrical central portion (112) having diameter of the transverse section analogous to that of the lower base of the cone represented by said conical portion (111), a catenoid-like pinched cylindrical portion (113) having a radius of curvature comprised between 12 mm and 14 mm, and said spherical end (130), the latter having radius of curvature comprised between 1 mm and 2 mm, said electrode (100) with spherical end (130) having an overall length comprised between 40 mm and 60 mm, said conical portion (111) having length comprised between 2 mm and 12 mm, said central cylindrical portion (112) having length comprised between 6 mm and 16 mm, said catenoid-like pinched cylindrical portion (113) having length comprised between 3 mm and 13 mm, said electrode (100) with spherical end (130) further being covered with biocompatible isolating mineral material.

17. Electrode (100) according to claim 16, further comprising: the flaring (122) having a diameter of 4.5 mm at 90°, the internal cavity (123) having width of 2.3 mm and length of 15 mm, the tail (120) having width of 8 mm and length of 25 mm, the conical portion (111) having a length of 7 mm, the central cylindrical portion (112) having a length of 11.31 mm, the catenoid-like pinched cylindrical portion (113) having a length of 8.69 mm and radius of curvature of 13.25 mm, the spherical end (130) having a radius of 1.5 mm, said electrode (100) with spherical end (130) having an overall length of 52 mm.

18. Electrode (100) comprising a tail (120) and a head (110), wherein said tail (120) is analogous to that of the electrode (100) according to claim 17, and wherein the head (110) is shaped to form a corkscrew-like element (140), the latter centrally defining the hole (142), said head (110) shaped to form a corkscrew-like element (140) defining, with the tail (120) of the electrode in which it is comprised, the symmetric lateral angles (141) comprised between 100° and 115°, said angles being observable when said hole (142) is parallel to the skin of the patient, said head (110) shaped to form a corkscrew-like element (140) further defining with said tail (120) the front/rear symmetric angles (143) comprised between 130° and 140°, said angles being visible when said hole is orthogonal to the skin of the patient, said head (110) shaped to form a corkscrew-like element (140) further having, for each side, at least one lateral curvature (144), at least one upper curvature (145), and at least one lower curvature (146), said electrode further being covered with biocompatible isolating material.

19. Electrode (100) according to claim 18, wherein the symmetric lateral angles (141) are 112.75° and in that the front/rear symmetric angles (143) are 135°.

20. Electrode (100) according to claim 16, wherein the electrode is covered with alumina or silicon nitride.

21. Electrode (100), covered with alumina or silicon nitride, with head (110) including the spherical end (130), according to claim 16 for use in the cold plasma treatment of skin blemishes.

22. Electrode (100), covered with alumina or silicon nitride, with head (110) shaped to form a corkscrew-like element (140) according to claim 18 for use in the cold plasma treatment of skin blemishes.
